(19) United States
(12) Patent Application Publication
(10) Pub. No.: US 2012/0165808 A1
(43) Pub. Date: Jun. 28, 2012

(54) Pulsed power laser actuated catheter system for interventional oncology

(76) Inventor: Adam Holiday Griffin, San Diego, CA (US)

(21) Appl. No.: 12/930,072

(22) Filed: Dec. 27, 2010

(51) Int. Cl. A61B 18/14

(52) U.S. Cl. 606/41

(57) ABSTRACT

Apparatus for delivering fast rising, brief pulsed electric fields of pre-determined duration into a catheter. Electrical current pulses of pre-determined duration and associated with cancer treatment using fiber coupled laser, switch, pre-charged transmission line and integrated catheter delivery system. The apparatus comprises a first stage pre-charged transmission line, fiber coupled to a short-pulse laser triggering a high voltage semiconductor switch with approximately 500 Amperes of photocurrent. The output of this first stage drives a second stage transmission line delivering up to approximately 2 Megawatts pulsed power into the catheter. The output of the second stage drives the final stage or catheter tip containing an electric field shaping functioning to treat cancerous tumors.

### CROSS-REFERENCE TO RELATED APPLICATIONS

United States Patent Application 20100228248

Kind Code A1

Griffin; Adam H. Sep. 9, 2010

Method and device for treating cancer with electrical therapy in conjunction with a catheter and high power pulser

### REFERENCE TO SEQUENCE LISTING, A TABLE, OR COMPUTER PROGRAM LISTING

None.

### BACKGROUND OF THE INVENTION

This invention relates to an apparatus for pulsed power light actuated catheters (PPLAC) and specifically for interventional oncology, whereby a laser actuated semiconductor switch launches and then delivers using a small catheter, a short pulse on the order of 1 nano-second pulse width and power levels capable of exceeding 2 Megawatts. Employing Silicon switching and a short pulse laser, a charged portion of the catheter is actuated using a high power laser for the purpose of pulsing cancerous tumors where ordinary catheter delivery systems would fail to produce such short pulses and high power levels. Pulsed Power Laser Actuated Catheters are part of a larger class of radio frequency developments related to oncology and can be used in a wide variety of cases where the physician has access to a catheter lab.

Recent inventions have examined the concept of treating cancerous tumors on the skin using pulsed electric fields. However doctors want tools that attack tumors within the body, and hence catheter type intervention is needed. Placing a “charged section” at the proximal end of a catheter then discharged down the catheter using a tiny high voltage switch accomplishes the method. The charged section stores energy electrostatically and couples to the catheter tip beginning the instant laser light impinges on the switch. The present invention addresses a new and unique method for controlling power catheters using a high voltage switch embedded within the catheter and light passing through its electrode. The invention employs standard technology for building catheters, and without having to rely on extraordinary high voltage materials. Furthermore, this invention provides for repetitive delivery of small amounts of electrical energy, inducing a repetitive high power pulsed electric field at the tip of the catheter.

The present invention is based on the realization that a reverse-biased thyristor or diode can be used to discharge a charged transmission line instantaneously when a picosecond class laser illuminates the switch. A catheter geometry lends itself to coaxial transmission line fabrication of the type described in this invention. Recent advances in the Silicon PIN diode fabrication allows direct illumination of reverse-biassed silicon through narrow windows patterned in the metallization. The picoseconds laser deposits photons into the silicon in a time substantially shorter than the discharge time of the transmission line, and therefore the rise time of the switch is governed by the laser pulse width. Commercial Nd:YAG lasers produce a single or repetitive 100 ps-200 ps FWHM pulse, and therefore a catheter transmission line was designed to deliver performance advantages including low conduction losses, fast rise and fall times, short pulse widths and power levels exceeding 2 Megawatts. Low on-resistance is accomplished using silver conductors, pulse widths are designed using the proximal 10 centimeters of the catheter, and power levels are amplified by the catheter geometry towards the distal end where treatment is made. Commercially available Silicon wafers have made this invention possible in addition to commercially available lasers, electrical components used to charge the catheters, and a number of catheter extrusion and braiding fabrication vendors.

This invention uses just one fiber coupled to a high power silicon PIN diode switch connected to a charged coaxial transmission line that is surrounded by a second transmission line along the entire 5 foot catheter length. The second transmission line is coupled to the first transmission line by virtue of being fabricated concentrically. The high power diode or thyristor switch is coupled to a laser and hence the diode acts as a switch when illuminated briefly by the laser.

The laser provides turn-on of a miniature high voltage semiconductor switch when it is used to actuate switching by the delivery of energetic photons to the switch. For example, a typical high power Silicon switch conducting 500 Amperes with rise and fall times less than 100 picoseconds and pulse width of 1 nanosecond would require laser-induced charge of 500 Amps*1 nanosecond=500 nano-Coulombs. Just one microjoule of laser energy at 1.064 micrometer wavelength (delivered in 100 picoseconds) is 860 nano-Coulombs. Therefore 100 kW peak power Nd:YAG laser pulse with 100 ps FWHM is sufficient to actuate the catheter switch. Commercially available pulsed Nd:YAG lasers suitable for this technology use stimulated brillouin scattering to achieve zero pre-pulse.

### SUMMARY OF THE INVENTION

The object of this invention is to provide a new and simple modality of launching a high power electrical pulse down a catheter using a high power laser and a high power silicon switch. The catheter is charged to a desired level e.g. from 100V to 10,000V prior to a laser pulse discharging the catheter. When the laser impinges into the catheter end a powerful yet low-energy electrical pulse is generated and travels near the speed of light towards the tip of the catheter where therapy is administered. When the pulse reaches the catheter tip, a high value pulsed electric field exists briefly for a duration of approximately one nanosecond. Subsequent charging and discharging of the catheter a pre-determined number of repetitions affects the treatment.

The present invention is designed to repetitively switch e.g. 500 amperes of pulsed current thus generating a powerful microwave using the catheter as a wave guide and pulsed electric field delivery vehicle. The present invention is based on the objective to operate at high voltage pulsed electric fields and allows specific delivery of low energy and high power pulses for cancerous tumor treatment.

### BRIEF DESCRIPTION OF THE DRAWINGS

Figure I shows one embodiment of a catheter system cross section according to the invention excluding the catheter tip.

Figure II shows the proximal end view of the catheter system shown in Figure I where the switch inputs and laser window are disclosed.

Figure III shows an end view of the catheter system shown in Figure I where the energy storage portion is disclosed and the physical dimensions are defined.

Figure IV shows an end view of the catheter system shown in Figure I where the pulse delivery portion is disclosed and the physical dimensions are defined.

Figure V is a schematic equivalent of the catheter system of Figure I including 25 Ohms approximation of a cancerous tissue near the catheter tip.

Figure VI is a simulated plot of power delivered to the cancerous tumor derived from Figure V for various conducting materials used in fabrication of the catheter system.

Figure VII is simulated high voltage pulse waveform delivered to the cancerous tumor derived from Figure V for various conducting materials used in fabrication of the catheter system.

Figure VIII is a simulated switching current derived from Figure V for various conducting materials used in fabrication of the catheter system.

Figure IX is a simulated high voltage pulse waveform delivered to the cancerous tumor derived from Figure V for two different Ohms approximation of a cancerous tissue near the catheter tip and using Silver conductors.

Figure X is a simulated maximum power level waveform exceeding 2 Megawatts delivered to the cancerous tumor derived from Figure V for 25 Ohms approximation of a cancerous tissue near the catheter tip and using Silver conductors.

Figure XI shows the distal cross section of the catheter system shown in Figure I where the tip used to shape the resulting pulsed electric field is fastened using a conformal radio opaque marker.

Figure XII shows the cross section of the catheter tip assembly of Figure XI.

### DETAILED DESCRIPTION OF THE PREFERRED EMBODIMENT

This invention provides a means for delivering pulsed electric fields effective in therapy of cancer tumors and the catheter system uses a laser, optical fiber, light actuated switching, a charged transmission line, a catheter waveguide delivery system and pulsed electric field shaping catheter tip. The invention conveys a preferred method to first employ short-pulse fiber-coupled laser light, second drive a charged transmission line repetitively in a system consisting of common off the shelf components assemblies and third employ a high-power silicon switch. The utility of this type of catheter system is the fast rise-time, narrow pulse widths and high power achievable with low component count.

High power light actuated catheter and short duration laser control of the type described by this invention using one fiber per catheter switch, employs moderate power pre-determined infrared laser pulse lengths e.g. with pulse widths in the range 100-200 pico-seconds discharging one transmission line into another and delivering a high power, short duration pulse to the catheter tip where treatment is performed. In the case of delivering pulsed electric fields to a cancerous tumor using the invention, electrical current is switched from the charged portion of the transmission line by the laser actuated switch.

Referring to Figure I, the cross section of the catheter system, there are two ground conductors, connections 2 and 6. The first transmission line conductor 4 is charged to high voltage, e.g. 100V up to 10,000V depending on the pulsed voltage desired to appear at the output of the catheter. Switch 1 is situated in region 8 between conductors 2 and 4. As a result of a short pulse laser being introduced into the volume of switch 1, a current flows through switch 1, and an electromagnetic wave travels out of region 9 and propagates into region 10 and towards the catheter tip (e.g. five feet further, into the cancerous tumor). The dielectric 3 and 5 of region 9 serves to store the initial electrostatic energy prior to the laser impingement and if this dielectric is made from e.g. FEP polymer it has a dielectric constant of 2.1 and voltage holding of between 70 kV/mm and 260 kV/mm for film thicknesses of 1 mil and 20 mil respectively (thin films hold more voltage than thick films due to reduced defect densities). For example the total stored capacitive energy in region 9 is 700 micro Joules when the switch is blocking 5000 Volts or half the maximum rating. Prior to the laser pulse arriving at the switch, energy is stored in dielectrics 3 and 5 through charging wire 12 with respect to ground conductors 2 and 6. Conducting member 11 serves to make an electrical connection from the switch to both inner conductor 2 and also to charging wire 12, and in this manner the central conductor 2 can be grounded. The entire structure is overcoated with a jacket 7, for example FEP. The conductors are preferably made from low-resistance braided Silver in regions 9 and 10, and the braid is expanded in region 8 to make connection to the switch. One simplistic way to understand how the energy stored in region 9 becomes an electromagnetic wave traveling into region 10 and ultimately to the catheter tip e.g five feet away is to think of the space in region 9 between conductors as filled with tiny discrete capacitors, and think of the conductors in region 9 connecting the tiny capacitors as made from tiny discrete inductors. As the current dumps from one capacitor to another through the switch and inductances a wave forms and propagates down the line. The electrical length of region 9 is a transmission line designed to be e.g. 0.5 nano-seconds when its physical length is 10 cm and the dielectric is FEP, and hence the wave that forms is 1.0 nano-second in length as is well known fact to those skilled in the art. The traveling electromagnetic wave emerges from region 9 into region 10 in a time 0.5 nano-second after the laser enters switch 1, and exits region 9 completely in a time 1.5 ns after the laser enters switch 1. These facts demonstrate the importance for the laser pulse width being much less duration than the transit time of the waves across region 9 so that the charge induced in the switch effectively shorts the switch before current begins to flow in the switch. The reader will appreciate using e.g. medical grade epoxy and silicone gel for assembly in the neighborhood of the switch, and non-lead based solders can be used to fasten connection block 11 and braided conductor 4 to the switch. Connecting wire 12 and center conductor 2 can be electrically fastened to conducting block 11 using e.g. non-lead based solders, welding, swaging or screw threads.

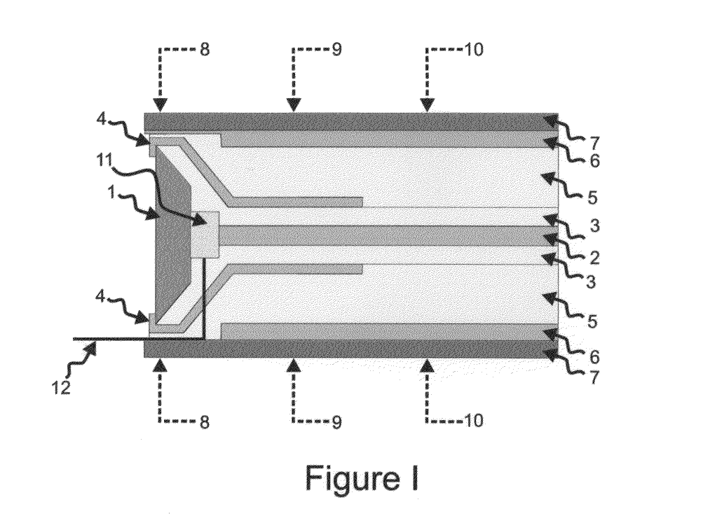

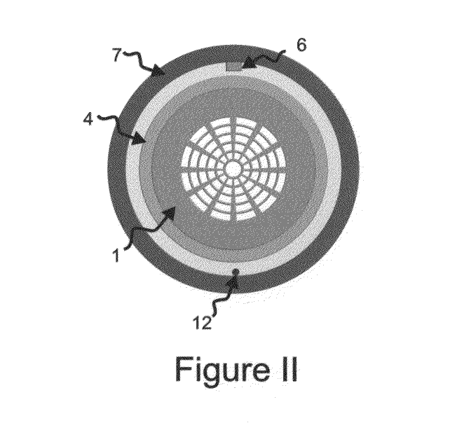

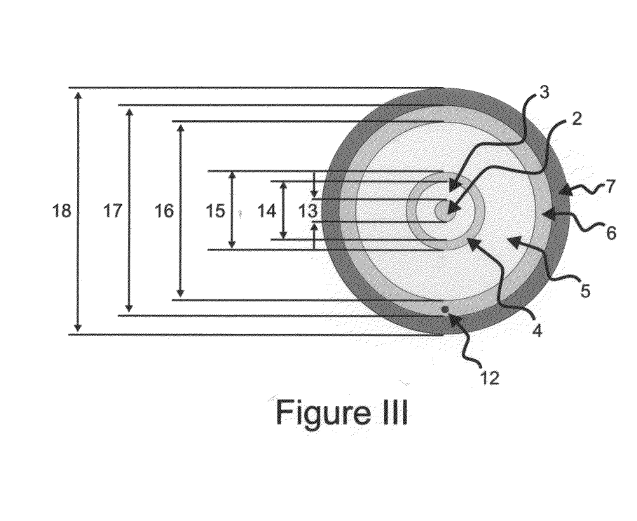

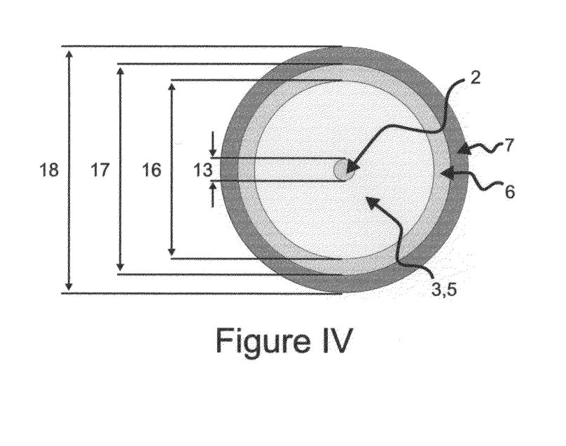

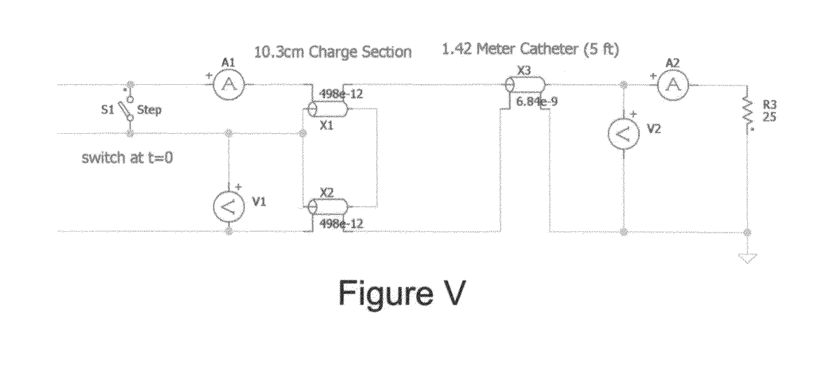

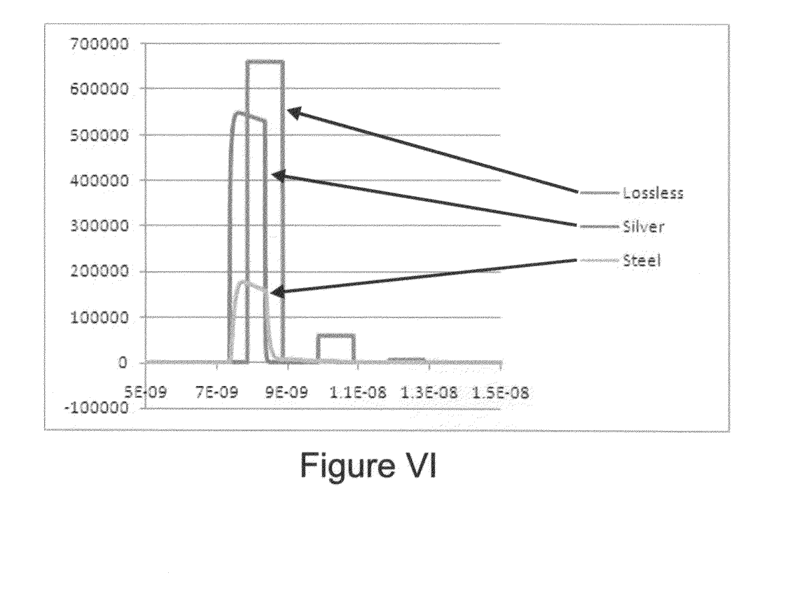

Referring to Figure II, Region 8 of Figure I is shown from the end view. Figure II describes the view the laser beam would have as it impinges on the switch. Switch 1 contains a multiplicity of narrow window designed to admit laser light through its conductor and enter the switch in pico-seconds. The window disclosed in switch 1 is where metal has been removed from the surface of the switch and this way laser light passes through the switch metallization and into the switch underneath. One microjoule of energy deposited in the switch in a time of 100 picoseconds is sufficient to allow the switch to conduct 500 Amperes of current for 1 nanosecond (stored energy in region 9 capacitance discharges through the switch and generates a traveling electromagnetic wave). Conductor 4 is electrically connected to the switch, whereas conductors 12 and 6 are electrically connected to ground. A voltage in the range e.g. 100 Volts and 10,000 Volts is repetitively applied across the switch (thus charging region 9 of FIG. 1) and then the laser pulse then repetitively arrives affecting transfer of energy down the catheter waveguide and towards its therapeutic tip. Insulating jacket 7 of diameter e.g. 1 mm shrouds the entire catheter and serves to strengthen and lubricate the catheter, it is extruded from e.g. FEP.

Figure III is an end view of the catheter shown in region 9 of Figure I where the electrostatic energy is stored. Dielectric region 3 is where the electrostatic energy is more dense, and dielectric region 5 is where the electrostatic energy is less dense (the electrostatic energy becomes a traveling energy wave once laser light actuates the switch and ultimately produces a high pulsed electric field at the catheter tip). Conductors 2, 4 and 6 are preferably made from silver wire and braid because the conduction losses are minimized in comparison with e.g. stainless steel. When the dielectric layers 3 and 5 are made from FEP the stored energy is e.g. 340 micro-Joules and 370 micro-Joules respectively when the system is charged to 5,000 Volts or half the rated voltage. Typical dimensions for diameters 13, 14, 15, 16, 17, 18 are 12 mils, 18 mils, 20 mils, 31 mils, 33 mils, 39 mils respectively. The electrical impedance of the portions of the catheter bounding dielectric regions 3 and 5 are e.g. 18 Ohms and 17 Ohms. The central silver conductor 2 is 12 mils diameter and the conducting ribbons 4,6 are 0.5 mil thickness (1 mil braided layers). The minimum dielectric thickness is defined to be 3 mils hence this catheter is manufacturable using standard extrusion and braiding technology. The materials slyer and FEP are biocompatible comprising the extruded and conducting stiffening portions of the catheter.

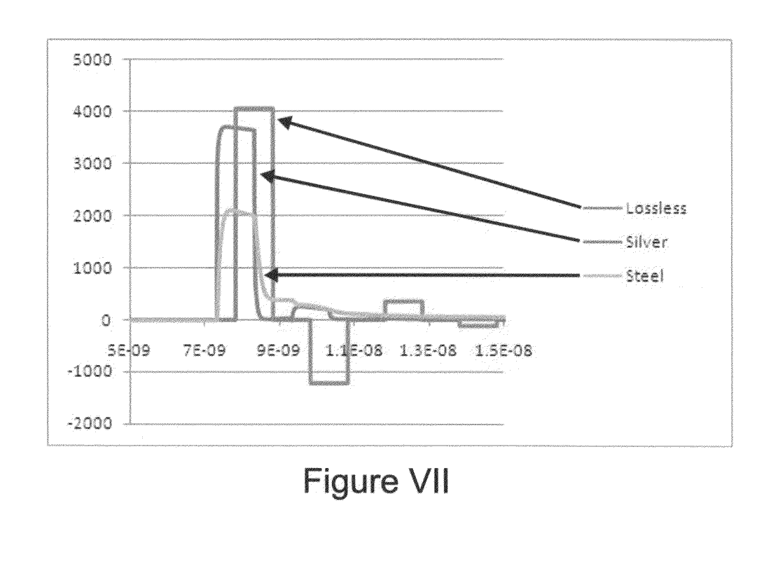

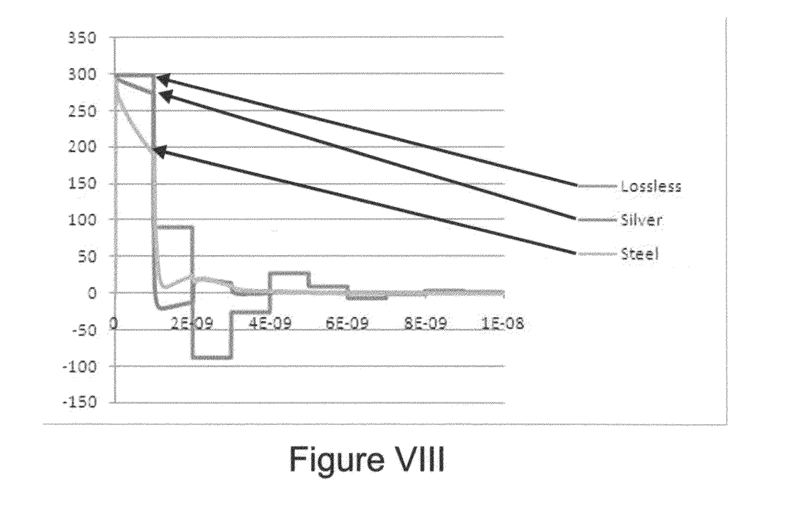

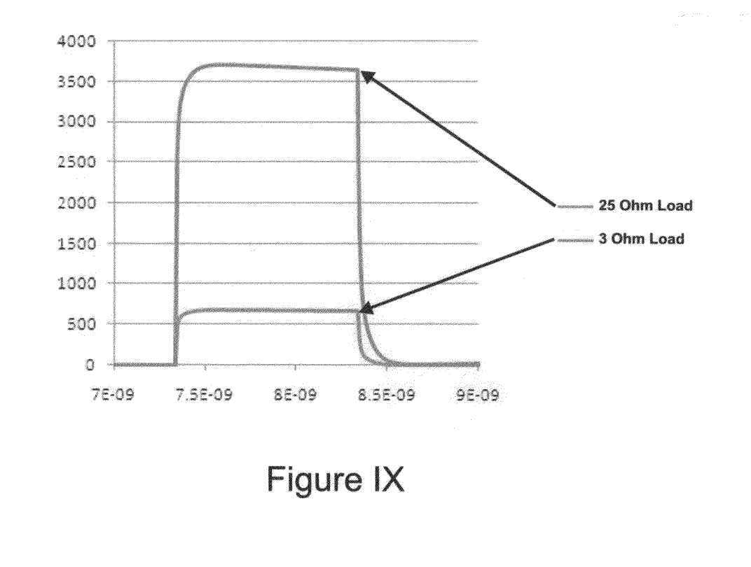

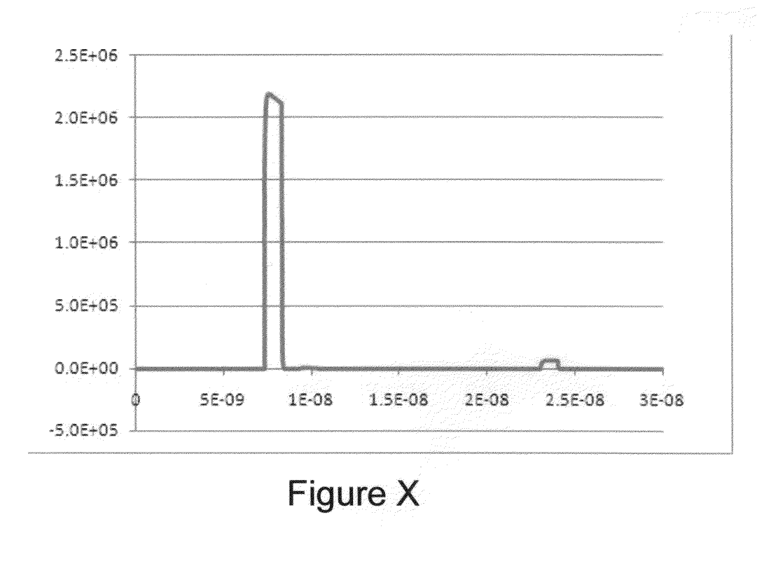

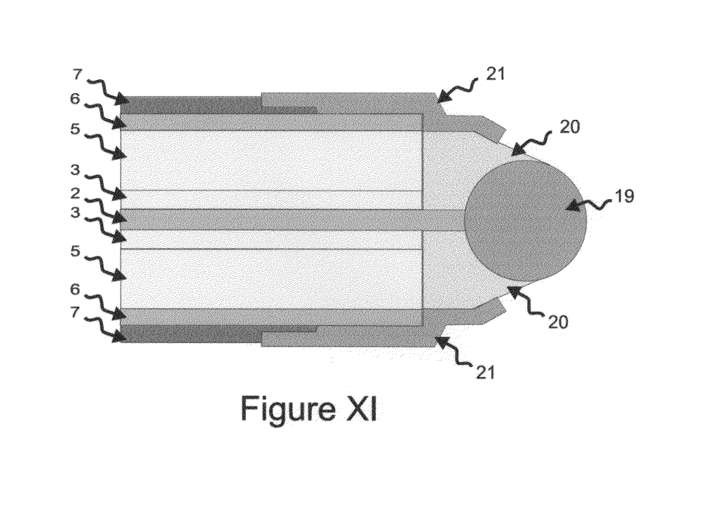

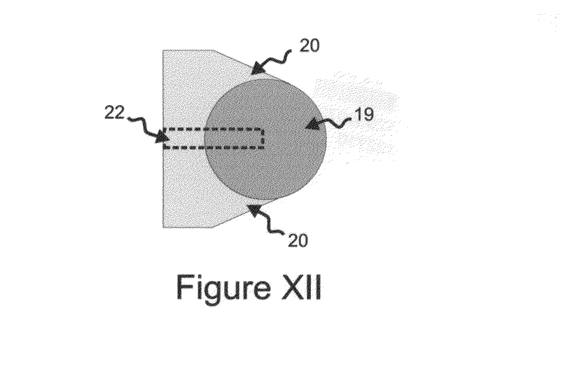

Figure IV is an end view of the catheter shown in Figure I region 10 where the electrostatic energy emitted from region 9 travels a long distance, e.g. five feet, to the catheter tip. Region 3,5 is where the electromagnetic energy is traveling and the current is flowing along conductors 2 and 6. Resistive losses is the conductors are minimized when these are preferably made from silver wire and braid because the conduction losses are minimized in comparison with e.g. stainless steel. Dielectric layers 3, 5 are made from e.g. FEP (dielectric layer 3 is extruded prior to metal braid layer 4 of FIG. 3, thereafter dielectric layer 5 is extruded on top of both). Typical dimensions for diameters 13, 16, 17, 18 are 12 mils, 31 mils, 33 mils, 39 mils respectively. The electrical impedance of this portion of the catheter is e.g. 39 Ohms. The silver core is e.g. 12 mils and the conductor 6 is comprised of e.g. 0.5 mil silver ribbon (1 mil braided layers). The minimum dielectric thickness is defined to be 3 mils hence this catheter is manufacturable using standard extrusion and braiding technology. The materials silver and FEP are biocompatible materials comprising the extruded portion of the catheter.

Figure V discloses the electrical circuit equivalent used to simulate the catheter of FIG. 1. It is modeled as two transmission lines X1 and X2 each with electrical length 498 pico-seconds according to their length and FEP dielectric. The long portion of the catheter X3 has an electrical length of 6.84 nano-seconds before being terminated at the catheter tip with a simple 25 Ohm model of human tissue estimated as follows: Published vales of resistivity for blood, muscle and fat are in the range 1.6 Ohm-meter, 3 Ohm-meter and 15 Ohm-meter respectively. At the e.g. 1 mm diameter catheter tip, the approximate working distance is e.g. 2 mm and a typical path length of e.g. 3 mm through an effective area of e.g. 2 square millimeters. Thus the effective impedance of these materials is in the range of 3 to 25 Ohms in which case both were simulated with 25 Ohms being closely matched to that of the catheter and hence the highest power is delivered. The lower impedance case results in lower power delivered to the tumor, but this just means the energy takes longer to exit the catheter while it is reflecting back and forth within the catheter due to any impedance mismatch at the tip.

Figure VI shows the simulated power delivered to a 25 Ohm load on the catheter tip and using no ohmic losses, losses in silver conductors and conduction losses in steel conductors. Clearly the lossy steel conductor is not a desirable conductor as the power is only 30 percent of that for silver. The axes on Figure VI are seconds on the ordinate and Watts on the abscissa. The charge is 5,000 Volts or half the rated value in this simulation and the resulting power delivered to the load is half a Megawatt.

Figure VII shows the simulated voltage delivered to a 25 Ohm load with no ohmic losses, conduction losses in silver conductor and conduction losses in steel conductor. Clearly the steel conductor is not a desirable conductor as the voltage is a little more than half that for silver. The axes on Figure VII are seconds on the ordinate and Volts on the abscissa. The charge is 5,000 Volts or half the rated value in this simulation and the resulting voltage delivered to the load is 3800 Volts.

Figure VIII shows the simulated switch current with the 25 Ohm load with ohmic losses, conduction losses in silver conductor and conduction losses in steel conductor. Clearly the steel braid is not a desirable conductor as the current droops significantly during switch conduction compared to that for silver. The axes on Figure VIII are seconds on the ordinate and Amperes on the abscissa. The charge is 5,000 Volts or half the rated value in this simulation and the resulting current passing through the switch is 280 Amperes.

Figure IX shows the simulated load voltage with both 25 Ohm and 3 Ohm load, using silver conductors. The pulse width is 1 nano-second in either case. The axes on Figure IX are seconds on the ordinate and Volts on the abscissa. The charge is 5,000 Volts or half the rated value in this simulation and the resulting voltage delivered is expected to be in the range of 700 Volts to 3700 Volts depending on the type of tumor being treated.

Figure X shows the simulated maximum load power with the 25 Ohm load and conduction losses in silver conductors. The axes on Figure X are seconds on the ordinate and Watts on the abscissa. The charge is 10,000 Volts or the full-rated value in this simulation and the brief 1 nano-second resulting power present across the load is 2.2 Megawatts.

Figure XI shows the integration of the catheter tip where the high voltage 1-nanosecond pulse exits the catheter. Center conductor 2 is connected to a spherical or rounded center conductor tip 19, and the electric field return lines terminate on conducting radio opaque marker 21. Insulating layer 20 prevents arcing between catheter tip conductor 19 and 21, serving to prevent the catheter tip from becoming detached. Radio opaque marker 21 further serves to make electrical connection between outer conducting silver braid 6. Radio opaque marker 21 can be tightly fit to the tip of the catheter using e.g. crimp or biocompatible adhesive.

Figure XII shows the construction of the catheter tip which has the purpose of shaping the electric field near or within a tumor situated at the catheter tip. The desired tip shape depends on the type and size of the tumor. The tip consists of a bio compatible conducting electrode 19 which may or may not be coated with a dielectric layer, and the tip is cast or molded within insulator 20. A hole 22 is also included for attachment of electrode 19 to the silver central electrode 2 shown in Figure XI. Attachment of the catheter tip assembly can be e.g. press-fit, soldered, threaded, or swaged to the catheter end.

What I claim as new and desire to protect by letters of patent of the United States is:

1. An apparatus for delivering high voltage, fast rise time and short duration electrical impulses using a charged transmission line switched inside a catheter microwave guide with a miniature high voltage semiconductor switch and laser, said catheter comprising an electrical circuit including:
pre-charged transmission line with charge introduced onto the central conducting member with respect to an inner conducting member and outer conducting member;
high voltage semiconductor switch between central conducting member and inner conducting member actuated by a short-pulse laser;
extended length of transmission line for transmitting the pulse emitted by the pre-charged transmission line and switch towards the tip of the catheter where the electrical impulse will deliver therapy to a patient; and
means for shaping the pulsed electric field at the catheter tip using a conducting central member contained within dielectric encapsulant and terminating a pulsed electric field on the radio opaque marker surrounding the catheter tip.

2. The apparatus according to claim 1, wherein said catheter of several feet length or more includes for insertion into an orifice or incision in the human body.

3. The apparatus according to claim 1, wherein said catheter includes a multiplicity of concentrically nested conductors with at least one conductor pre-charged and actuated using a laser and semiconductor switch.

4. The apparatus according to claim 1, wherein said switching means includes a semiconductor switch (thyristor or diode) with a multiplicity of narrow window openings in the metalization for depositing charge into the semiconductor using a fiber-coupled short pulse laser.

5. The apparatus according to claim 1, wherein said catheter tip includes a means for shaping the pulsed electric field at the tip of the catheter.
